(19) United States
(12) Patent Application Publication
(10) Pub. No.: US 2005/0020671 A1
(43) Pub. Date: Jan. 27, 2005

(54) Pharmaceutical formulation

(76) Inventors: Matilde Fernandez Ibanez, Madrid (ES); Emilio Garriz Sanz, Madrid (ES)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/467,504

(22) PCT Filed: Feb. 13, 2002
(86) PCT No.: PCT/EP02/01668

(30) Foreign Application Priority Data
Feb. 14, 2001 (ES) P200100329

(51) Int. Cl. A61K 31/34; A61K 9/24

(52) U.S. Cl. 514/471; 424/472

(57) ABSTRACT

The present invention provides a pharmaceutical formulation which comprises a core comprising a first active ingredient, a coating comprising a second active ingredient which is incompatible with the first active ingredient and a barrier between the core and the coating which prevents physical contact between the core and the coating, characterised in that the barrier is formed on the core by film-coating and the coating is formed on the barrier by press-coating.

The present invention relates to improvements in the formulation of pharmaceutical compositions. In particular, it relates to improvements in the formulation of pharmaceutical compositions wherein two or more active ingredients are present in the composition and wherein at least two of the active ingredients are incompatible with one another.

In the context of the present application, the term “incompatible”, when applied to active ingredients, means that the active ingredients cannot normally be formulated into a pharmaceutical composition wherein the active ingredients are in physical contact. The incompatibility is usually, but not necessarily, the result of a chemical reaction between the active ingredients which results in a reduction of the therapeutic activity of at least one of the active ingredients.

It will be appreciated by a person skilled in the art that many pharmaceutical compositions which comprise incompatible active ingredients may be formulated as described herein. However, the present invention is particularly directed to compositions which comprise acetylsalicylic acid or a physiologically acceptable salt of acetylsalicylic acid and ranitidine or a physiologically acceptable salt of ranitidine, such as ranitidine hydrochloride.

Systemic non-steroidal anti-inflammatory drugs such as acetylsalicylic acid are known to give undesirable side effects. In particular they are known to be ulcerogenic and can therefore give rise to gastric ulceration when administered orally over a period of time to a patient.

Ranitidine is the approved name for N-[2-[[[5-(dimethylamino)methyl]-2-furanyl]methyl]thio]ethyl-N′-methyl-2-nitro-1,1-ethanediamine which is described and claimed in British Patent No 1,565,966. It is known to be a potent histamine H₂-antagonist which may be used in the treatment of conditions where there is an advantage in lowering gastric acidity, particularly in gastric and peptic ulceration.

It is also known from British Patent No 2,105,193 that mucosal lesions of the gastrointestinal tract caused by systemic non-steroidal anti-inflammatory drugs can be significantly reduced by co-administering ranitidine. British Patent No 2,105,193 discloses pharmaceutical compositions comprising a systemic non-steroidal anti-inflammatory drug and ranitidine or a physiologically acceptable salt thereof. However, experiments show that there is a clear chemical incompatibility between ranitidine hydrochloride (the most preferred physiologically acceptable salt of ranitidine) and acetylsalicylic acid. These two active ingredients react chemically with each other to produce degradation products. The reaction becomes manifest in a matter of days and implies a serious stability problem for pharmaceutical compositions containing both active ingredients.

The present invention addresses the problems outlined above and provides a pharmaceutical formulation which comprises a core comprising a first active ingredient, a coating comprising a second active ingredient which is incompatible with the first active ingredient and a barrier between the core and the coating which prevents physical contact between the core and the coating, characterised in that the barrier is formed on the core by film-coating and the coating is formed on the barrier by press-coating.

It will be appreciated by those skilled in the art that further barriers and coatings may be formed on the composition if so desired. This may be necessary if, for example, more than two active ingredients are present and each active ingredient is incompatible with all or some of the other active ingredients.

In one embodiment of the present invention the core comprises ranitidine or a physiologically acceptable salt thereof and the coating comprises acetylsalicylic acid or a physiologically acceptable salt thereof.

In a preferred embodiment, ranitidine or a physiologically acceptable salt thereof is present in the core in an amount sufficient to reduce gastrointestinal distress caused by acetylsalicylic acid. In a more preferred embodiment, ranitidine is present in the core in an amount of from 10 to 200 mg or a physiologically acceptable salt of ranitidine is present in the core in an amount which is equivalent to from 10 to 200 mg of ranitidine. In a more preferred embodiment, ranitidine is present in the core in an amount of from 50 to 100 mg or a physiologically acceptable salt of ranitidine is present in the core in an amount which is equivalent to from 50 to 100 mg of ranitidine. In a more preferred embodiment, ranitidine is present in the core in an amount of from 70 to 80 mg or a physiologically acceptable salt of ranitidine is present in the core in an amount which is equivalent to from 70 to 80 mg of ranitidine.

It is preferable that the ranitidine be present in the form of a physiologically acceptable salt. Such salts include salts of inorganic or organic acids such as the hydrochloride, hydrobromide, sulphate, acetate, maleate, succinate and fumarate salts. In a particularly preferred embodiment, ranitidine is present in the form of ranitidine hydrochloride.

As well as the first active ingredient, the core may also comprise one or more pharmaceutical excipients, disintegrants, lubricants, anti-adhesion agents, flow agents, diluents or taste-masking agents.

In a preferred embodiment, the one or more lubricants are present in the core in an amount of from 0.1 to 5% by weight based on the total weight of the core. In a more preferred embodiment, the one or more lubricants are present in the core in an amount of from 0.1 to 3% by weight based on the total weight of the core. In a still more preferred embodiment, the one or more lubricants are present in the core in an amount of from 0.5 to 1.0% by weight based on the total weight of the core. Examples of suitable lubricants include magnesium stearate, calcium stearate, zinc stearate, stearic acid, palmitic acid and sodium stearyl fumarate. A preferred lubricant is magnesium stearate.

In a preferred embodiment, the one or more diluents are present in the core in an amount of from 10 to 90% by weight based on the total weight of the core. In a more preferred embodiment, the one or more diluents are present in the core in an amount of from 30 to 70% by weight based on the total weight of the core. In a still more preferred embodiment, the one or more diluents are present in the core in an amount of from 40 to 60% by weight based on the total weight of the core. Examples of suitable diluents include microcrystalline cellulose, powdered cellulose, lactose, mannitol, sucrose and calcium phosphate. A preferred diluent is microcrystalline cellulose.

In a preferred embodiment, the one or more disintegrants are present in the core in an amount of from 0.1 to 10% by weight based on the total weight of the core. In a more preferred embodiment, the one or more disintegrants are present in the core in an amount of from 0.1 to 5% by weight based on the total weight of the core. In a still more preferred embodiment, the one or more disintegrants are present in the core in an amount of from 1 to 3% by weight based on the total weight of the core. An example of a suitable disintegrant is sodium croscaramelose.

In a preferred embodiment, acetylsalicylic acid is present in the coating in an amount of from 50 to 1000 mg or a physiologically acceptable salt of acetylsalicylic acid is present in the core in an amount which is equivalent to from 50 to 1000 mg of acetylsalicylic acid. In a more preferred embodiment, acetylsalicylic acid is present in the coating in an amount of from 300 to 700 mg or a physiologically acceptable salt of acetylsalicylic acid is present in the coating in an amount which is equivalent to from 300 to 700 mg of acetylsalicylic acid. In a more preferred embodiment, acetylsalicylic acid is present in the coating in an amount of from 450 to 550 mg or a physiologically acceptable salt of acetylsalicylic acid is present in the coating in an amount which is equivalent to from 450 to 550 mg of acetylsalicylic acid. In an embodiment the acetylsalicylic acid or the physiologically acceptable salt of acetylsalicylic acid may be microencapsulated with ethyl cellulose. An example of a commercially available microencapsulated acetylsalicylic acid product is Rhodine NCR-P™.

As well as the second active ingredient, the coating may also comprise one or more pharmaceutical excipients, disintegrants, lubricants, anti-adhesion agents, flow agents, diluents or taste-masking agents.

In a preferred embodiment, the one or more diluents are present in the coating in an amount of from 5 to 90% by weight based on the total weight of the coating. In a more preferred embodiment, the one or more diluents are present in the coating in an amount of from 5 to 50% by weight based on the total weight of the coating. In a still more preferred embodiment, the one or more diluents are present In the coating in an amount of from 10 to 20% by weight based on the total weight of the coating. Examples of suitable diluents include copovidone, microcrystalline cellulose, powdered cellulose, maize starch, lactose, Cellactose™80 (a compound formed from 25% powdered cellulose and 75% lactose monohydrate) and Ludipress™ (a compound formed from 93% lactose monohydrate, 3.5% povidone and 3.5% crospovidone). A preferred diluent is Cellactose™80.

In a preferred embodiment, the one or more disintegrants are present in the coating in an amount of from 0.1 to 10% by weight based on the total weight of the coating. In a more preferred embodiment, the one or more disintegrants are present in the coating in an amount of from 0.1 to 5% by weight based on the total weight of the coating. In a still more preferred embodiment, the one or more disintegrants are present in the coating in an amount of from 1 to 3% by weight based on the total weight of the coating. Examples of suitable disintegrants include maize starch, crospovidone, sodium starch glycolate, bentonite, aluminium magnesium silicate and sodium croscaramelose. A preferred disintegrant is sodium croscaramelose.

In a preferred embodiment, the one or more lubricants are present in the coating in an amount of from 0.1 to 10% by weight based on the total weight of the coating. In a more preferred embodiment, the one or more lubricants are present in the coating in an amount of from 0.1 to 5% by weight based on the total weight of the coating. In a still more preferred embodiment, the one or more lubricants are present in the coating in an amount of from 0.1 to 1.0% by weight based on the total weight of the coating. Examples of suitable lubricants include hydrogenated vegetable oil, stearic acid, palmitic acid and sodium stearyl fumarate. A preferred lubricant is sodium stearyl fumarate.

In a preferred embodiment, the one or more anti-adhesion agents are present in the coating in an amount of from 0.1 to 10% by weight based on the total weight of the coating. Examples of suitable anti-adhesion agents are talc and hydrogenated vegetable oil.

In a preferred embodiment, the one or more flow agents are present in the coating in an amount of from 0.01 to 5% by weight based on the total weight of the coating. In a more preferred embodiment, the one or more flow agents are present in the coating in an amount of from 0.01 to 1% by weight based on the total weight of the coating. In a still more preferred embodiment, the one or more flow agents are present in the coating in an amount of from 0.01 to 0.5% by weight based on the total weight of the coating. An example of a suitable flow agent is colloidal anhydrous silica.

The barrier is present in order to prevent physical contact between the core and the coating and therefore prevent reaction between the first and second active ingredients which are present in the core and the coating respectively. Preferably the barrier also reduces the transmission of moisture between the core and the coating. More preferably the barrier is substantially impermeable to the transmission of moisture between the core and the coating.

In a preferred embodiment the barrier is present in an amount of from 1 to 20% by weight based on the total weight of the core. In a more preferred embodiment the barrier is present in an amount of from 3 to 15% by weight based on the total weight of the core. In a still more preferred embodiment the barrier is present in an amount of from 5 to 10% by weight based on the total weight of the core.

In general terms any material that reduces water transmission and is compatible with the active ingredients contained in the core and the coating can be used to physically separate them. Thus, a barrier material which comprises one or more of stearic acid, polyvinyl alcohol, ethyl cellulose, microcrystalline cellulose, hydroxypropyl cellulose and methacrylic acid copolymer type C, can be used.

An example of a suitable barrier material is OPADRY™ Aqueous Moisture Barrier (AMB) produced by the Colorcon Company (Reference OY-B-28920). OPADRY™ AMB is made up of polyvinyl alcohol, titanium dioxide, purified talc, lecithin and xanthan gum. The formula is supplied as a powder which is reconstituted using purified cold water before it is sprayed on the core. The actual solids content of the coating suspensions can be varied according to the atomising capabilities of the spraying equipment. Preferably, the solids content is approximately 20% w/w.

Another example of a suitable barrier material is SEPIFILM™ Low Permeability (LP) produced by Seppic. SEPIFILM™ LP is made up of hydroxypropylmethyl cellulose, microcrystalline cellulose, stearic acid and pigments and/or lakes (if required).

Another example of a suitable barrier material is LUSTRECLEAR™ produced by FMC Corporation. LUSTRECLEAR™ is made up of microcrystalline cellulose, carrageenan, polyethylene glycol, hydroxyethyl cellulose, maltodextrin and pigments and/or lakes (if required).

Another example of a suitable barrier material is EUDRAGIT™ L 30D-55 produced by Degussa. If this barrier material is chosen, it should not be applied in an amount greater than approximately 1 mg/cm² of barrier material on the tablet surface. If more is used there is a danger of forming an enteric coating around the core which could reduce the bioavailability of the active ingredient in the core. EUDRAGIT™ L 30D-55 is made up of methacrylic acid—ethyl acrylate copolymer as a 30% dispersion in water. The mean relative molecular mass of the copolymer is approximately 250 000 and the ratio of carboxylic groups to ester groups is approximately 1:1. The material may also comprise surface-active agents such as sodium dodecyl sulphate and polysorbate 80. It contains not less than 46.0% m/m and not more than 50.6% m/m of methacrylic acid units, calculated with reference to the residue on evaporation.

Methods of manufacturing by press coating, also known as dry coating or compression coating are well known in the pharmaceutical industry. See for example; Pharmaceutics, the Science of Dosage Form Design, edited by M E Aulton, Published by Churchill Livingstone (part of the Longman Group) 1988, ISBN 0-443-03643-8. In particular pages 675 to 676 explain the method of press coating as follows. Press coating involves the compaction of granular material around an already preformed core using compressing equipment similar to that used for the core itself, e.g. Manesty Drycota. It is used mainly to separate chemically incompatible materials, one or more being placed in the core and the other(s) in the coating layer. However, there is still an interface of contact left between the two layers. In cases where even this is important then the process of press coating can be taken one step further. It is possible to apply two press coatings to a tablet core using suitable equipment, e.g. Manesty Bicota. This equipment produces press coated tablets with perfect separation between the active core and the coating as these two can be separated by an inert coating. The technique is also described in Reminton: The Science and Practice of Pharmacy 19^{th} Edition 1995, published by Mack Publishing Company, ISBN 0-912734-04-3. See in particular pages 1616 and 1631 which explain the method of manufacture by press coating as follows. Press coated tablets, also referred to as dry-coated, are prepared by feeding previously compressed tablets into a special tableting machine and compressing another granulation layer around the preformed tablets. They have all the advantages of compressed tablets, i.e. slotting, monogramming, speed of disintegration, etc, while retaining the attributes of sugar-coated tablets in masking the taste of the taste drug substance in the core tablets. An example of a press-coated tablet press is the Manesty Drycota. Press coated tablets can also be used to separate incompatible drug substances; in addition, they can provide a means to give an enteric coating to the core tablets.

The following journal articles also provide some discussion of the technique of press-coating:

Press-coated tablets for the sequential pulsed administration of two different drugs, International Journal of Pharmaceutics, Volume 99, Issues 2-3, 1993, pages 173-179, Maggi L, et al.

Press-coated tablets for time-programmed released of drug, Biomaterials, Volume 14, Issue 13,1993 1017-1023, Conte U, et al.

The technique of film-coating is also well known in the pharmaceutical industry and is described for example in Pharmaceutics, the Science of Dosage Form Design, edited by M E Aulton, Published by Churchill Livingstone (part of the Longman Group) 1988, ISBN 0-443-03643-8. In particular pages 672 to 675 explain the method of film coating as follows. Film coating involves the deposition, usually by a spray method, of a thin film of polymer surrounding the tablet core. It is possible to utilize conventional panning equipment but more usually specialized equipment is employed to take advantage of fast coating times and a high degree of automation is possible. The coating solution contains a polymer in a suitable liquid medium together with other ingredients such as pigments and plasticizers. This solution is sprayed on to a rotated mixed tablet bed. The drying conditions permit the removal of the solvent so as to leave a thin deposition of coating material around each tablet core. Typically the coating solution formulation comprises: polymer, solvent, plasticizer and colorants. The technique is also described in Reminton: The Science and Practice of Pharmacy 19^{th} Edition 1995, published by Mack Publishing Company, ISBN 0-912734-04-3. See in particular pages 1652 to 1659 which explain the method of film coating as follows. Film coating involves the deposition of a thin, but uniform, film onto the surface of the substrate. Unlike sugar coating, the flexibility afforded in film coating allows additional substrates other than just compressed tablets, to be considered (eg, powders, granules, nonpareils, capsules). Coatings are essentially applied continuously to a moving bed of material by means of a spray technique, although manual application procedures have also been used.

The following are examples of formulations which are representative of the present invention. They are intended to illustrate the invention but are not intended to be limiting on the scope of the invention which is defined by the claims.

### EXAMPLE 1

A double tablet having the following specifications for the core, barrier and coating was manufactured. The first active ingredient is ranitidine hydrochloride and the second active ingredient is acetylsalicylic acid:

Core

| | Weight per | % w/w (based on | |
|---|---|---|---|
| Core Ingredient | tablet (mg) | total core weight) | Function |
| Granulated | 84.000 | 56.00 | Active Ingredient |
| Ranitidine | | | |
| Hydrochloride | | | |
| Microcrystalline | 64.875 | 43.25 | Diluent |
| Cellulose | | | |
| Magnesium | 1.125 | 0.75 | Lubricant |
| Stearate | | | |

Barrier

| Barrier | Weight per | % w/w (based on | |
|---|---|---|---|
| Ingredient | tablet (mg) | total core weight) | Function |
| Opadry AMB ™ | 12.000 | 8.00 | Moisture Barrier |

Coating

| | | % w/w (based on | |
|---|---|---|---|
| Coating | Weight per | total coating | |
| Ingredient | tablet (mg) | weight) | Function |
| Acetylsalicylic | 500.000 | 82.40 | Active Ingredient |
| acid | | | |
| Cellactose ™ 80 | 91.020 | 15.00 | Diluent |
| Sodium | 12.140 | 2.00 | Disintegrant |
| Croscaramelose | | | |
| Sodium Stearyl | 3.030 | 0.50 | Lubricant |
| Fumarate | | | |
| Colloidal | 0.610 | 0.10 | Flow Promoter |
| Anhydrous | | | |
| Silica | | | |

The manufacturing method was as follows, the steps being numbered sequentially:

A) Manufacture of the Ranitidine Tablet Cores:

Mixture of the Ranitidine Tablet Core Ingredients

1) The selected amount of granulated ranitidine hydrochloride, microcrystalline cellulose and magnesium stearate were weighed out according to the batch size to be manufactured.

2) The granulated ranitidine hydrochloride and the microcrystalline cellulose were added to a drum blender or equivalent mixing equipment.

3) The mixture was blended for 10 minutes at a speed of rotation of 15 rpm to provide a homogenous mixture. Note that the mixing parameters may be varied according to the equipment used and batch size intended for manufacture.

4) To the homogenous mixture of ranitidine hydrochloride and microcrystalline cellulose was added the magnesium stearate. Mixing was continued in the same drum blender or equivalent mixing equipment for 5 minutes at a speed of rotation of 15 rpm. Again, the mixing parameters may be varied according to the equipment used and batch size intended for manufacture.

Compression of the Powder Mixture to Produce the Ranitidine Tablet Core

5) A tableting machine was used to compress the previous powder mixture into tablet cores in compliance with the following specifications:

| | |
|---|---|
| Weight: | 150 mg |
| Uniformity of Mass: | In compliance with European Pharmacopeia |
| Hardness: | greater than 8 Kp |
| Friability: | less than 1% w/w |
| Disintegration time: | less than 15 minutes |

The punches used were standard concave, 8 mm diameter.

B) Film-Coating the Ranitidine Tablet Cores:

6) The coating suspension of Opadry AMB was prepared as follows. The required amount of Opadry AMB and purified water to prepare a 20% w/w dispersion of Opadry AMB in purified water were measured out. Note that the recommended solids level is 20% w/w, but the actual solids level in the suspension can be changed to allow for the atomising capabilities of the spraying equipment. Opadry AMB was reconstituted as follows. The total quantity of cold water was poured into a suitable container. A propeller or similar type of stirrer was placed in the water, and rotated so that a vortex was produced, but without drawing air into the liquid. The Opadry AMB powder was added to the vortex as quickly as possible so that undispersed powder did not float on the surface of the liquid. During the addition step, the suspension viscosity rose, thus it was necessary to increase the stirrer speed in order to maintain the vortex. After all the Opadry AMB powder had been added, the stirrer speed was reduced until the vortex was nearly eliminated and stirring continued for a further 45 minutes, after which time the coating suspension was ready for use. It was preferable to provide gentle agitation to the coating suspension while it was being sprayed.

7) The selected amount of ranitidine tablet cores were placed into a suitable film coating equipment e.g. ACCELA-COTA or similar equipment. 12 mg of Opadry AMB film was coated on the ranitidine tablet cores using the following film coating parameters:

| | |
|---|---|
| Inlet air temperature: | ≧90° C. |
| Product temperature before spraying: | ≧70° C. |
| Drum speed: | 10 rpm |
| Spray equipment: | Manesty spray gun |
| Atomising air pressure: | 3.5 Bar |

The spraying parameters may be varied according to the equipment used and batch size intended for manufacture.

C) Press Coating the Ranitidine Film Coated Cores:

Mixture of the Acetyl Salicylic Acid (ASA) Tablet Coat Ingredients

8) The selected amounts of ASA, Cellactose™ 80, sodium croscaramelose, sodium stearyl fumarate and colloidal anhydrous silica were weighed out.

9) The ASA, Cellactose™ 80, sodium croscaramelose, sodium stearyl fumarate and colloidal anhydrous silica were added to a drum blender or equivalent mixing equipment.

10) The mixture was blended for 10 minutes at 15 rpm to obtain a homogenous mixture. Note that these parameters are Intended as a guide only. The proper parameters will depend upon the equipment and the batch size intended for manufacture.

Press Coating the ASA Powder Mixture onto the Film Coated Ranitidine Cores

11) A tableting machine which is capable of producing double compressed tablets e.g. Manesty Drycota or similar equipment was used to provide a tablet in compliance with the following specifications:

| | |
|---|---|
| Weight: | 768.8 mg |
| Uniformity of Mass: | compliant with European Pharmacopeia |
| Hardness: | greater than 10 Kp |
| Friability: | less than 1% w/w |

The punches used were standard concave, 12 mm diameter

The press coating process can be summarised in the following steps. Approximately half of the ASA powder mixture was filled into a tablet die. Then the filmed-coated ranitidine tablet was fed into the tablet die. Afterwards, the rest of the ASA mixture was filled into the die. The punches compressed the ASA coat onto the filmed-coated ranitidine tablet, producing a double compressed tablet where the tablet core is the filmed-coated ranitidine tablet and the external coat is the ASA. This cycle was repeated by the tableting machine as more film coated ranitidine tablets and ASA mixture (dry coating material) were fed into the dies from the hoppers.

This formulation was subjected to a stability study under the following conditions: 25° C./60% relative humidity and 40° C./75% relative humidity for 1, 2, 3 and 6 months. It was found that the ranitidine remains stable for 6 months at 25° C./60% relative humidity and at 40° C./75% relative humidity. The acetylsalicylic acid is stable for 6 months at 25° C./60% relative humidity and is stable for 2 months at 40° C./75% relative humidity.

### EXAMPLE 2

A double tablet having the following specifications for the core, barrier and coating was manufactured:

Core

| | Weight per | % w/w (based on | |
|---|---|---|---|
| Core Ingredient | tablet (mg) | total core weight) | Function |
| Granulated | 84.000 | 56.00 | Active Ingredient |
| Ranitidine | | | |
| Hydrochloride | | | |
| Microcrystalline | 64.875 | 43.25 | Diluent |
| Cellulose | | | |
| Magnesium | 1.125 | 0.75 | Lubricant |
| Stearate | | | |

Barrier

| Barrier | Weight per | % w/w (based on | |
|---|---|---|---|
| Ingredient | tablet (mg) | total core weight) | Function |
| Opadry AMB ™ | 12.000 | 8.00 | Moisture Barrier |

Coating

| | | % w/w (based on | |
|---|---|---|---|
| Coating | Weight per | total coating | |
| Ingredient | tablet (mg) | weight) | Function |
| Acetylsalicylic | 507.6-526.3⁽¹⁾ | 82.7-85.7 | Active Ingredient |
| Acid micro- | | | |
| encapsulated | | | |
| with Ethyl | | | |
| Cellulose | | | |
| (Rhodine NCR- | | | |
| P ™) | | | |
| Cellactose ™ 80 | 73.7-92.4⁽²⁾ | 12.0-15.0 | Diluent |
| Sodium | 12.3 | 2.0 | Disintegrant |
| Croscaramelose | | | |
| Sodium Stearyl | 1.2 | 0.2 | Lubricant |
| Fumarate | | | |
| Colloidal | 0.6 | 0.1 | Flow Promoter |
| Anhydrous | | | |
| Silica | | | |

(1) The purity of acetylsalicylic acid micro-encapsulated with ethyl cellulose varies between 95% and 98.5% w/w. Therefore the quantity of acetylsalicylic acid micro-encapsulated with ethyl cellulose that has to be included in each tablet can vary between 507.6 and 526.3 mg in order to provide a dose of 500 mg of acetylsalicylic acid.

(2) The amount of Cellactose™ 80 added can vary between 73.7 and 92.4 mg so as to provide a total of 600 mg for the combined weight of Cellactose™ 80 and acetylsalicylic acid micro-encapsulated with ethyl cellulose.

The manufacturing method was the same as for Example 1 above except that the weight of acetylsalicylic acid micro-encapsulated with ethyl cellulose has to be adjusted to provide 500 mg of acetylsalicylic acid and the difference in the final tablet weight is compensated with the amount of Cellactose™ 80.

This formulation was subjected to a stability study under the following conditions: 25° C./60% relative humidity and 40° C./75% relative humidity for 1, 2, 3 and 6 months. It was found that the ranitidine remains stable for 6 months at 25° C./60% relative humidity and at 40° C./75% relative humidity. The acetylsalicylic acid is stable for 6 months at 25° C./60% relative humidity and is stable for 2 months at 40° C./75% relative humidity.

1. A pharmaceutical formulation which comprises a core comprising a first active ingredient, a coating comprising a second active ingredient which is incompatible with the first active ingredient and a barrier between the core and the coating which prevents physical contact between the core and the coating, characterised in that the barrier is formed on the core by film-coating and the coating is formed on the barrier by press-coating.

2. A pharmaceutical formulation as claimed in claim 1 wherein the core further comprises one or more pharmaceutical excipients, disintegrants, lubricants, anti-adhesion agents, flow agents or diluents.

3. A pharmaceutical formulation as claimed in claim 1 wherein the coating further comprises one or more pharmaceutical excipients, disintegrants, lubricants, anti-adhesion agents, flow agents or diluents.

4. A pharmaceutical formulation as claimed in claim 1 wherein the core comprises ranitidine or a physiologically acceptable salt thereof and the coating comprises acetylsalicylic acid or a physiologically acceptable salt thereof.

5. A pharmaceutical formulation as claimed in claim 4 wherein the ranitidine is present in the core in an amount of from 10 to 200 mg or a physiologically acceptable salt of ranitidine is present in the core in an amount which is equivalent to from 10 to 200 mg of ranitidine.

6. A pharmaceutical formulation as claimed in claim 4 wherein the ranitidine is present in the form of ranitidine hydrochloride.

7. A pharmaceutical formulation as claimed in claim 4 wherein acetylsalicylic acid is present in the coating in an amount of from 50 to 1000 mg or a physiologically acceptable salt of acetylsalicylic acid is present in the core in an amount which is equivalent to from 50 to 1000 mg of acetylsalicylic acid.

8. A pharmaceutical formulation as claimed in claim 4 wherein the acetylsalicylic acid or the physiologically acceptable salt of acetylsalicylic acid is microencapsulated with ethyl cellulose.

9. A pharmaceutical formulation as claimed in claim 1 wherein the barrier also reduces the transmission of moisture between the core and the coating.

10. A pharmaceutical formulation as claimed in claim 1 wherein the barrier is present in an amount of from 1 to 20% by weight based on the total weight of the core.
